(19) United States
(12) Patent Application Publication
(10) Pub. No.: US 2024/0393263 A1
(43) Pub. Date: Nov. 28, 2024

(54) POLLUTION MONITORING USING INTEGRATED SENSING AND COMMUNICATION

(71) Applicant: RAKUTEN SYMPHONY, INC., Tokyo (JP)

(72) Inventor: Manmeet Singh BHANGU, Indore (IN)

(73) Assignee: Rakuten Symphony India Private Limited, Indore, MP (IN)

(21) Appl. No.: 18/271,891

(22) PCT Filed: Jun. 29, 2023
(86) PCT No.: PCT/US2023/026534

§ 371 Date: Jul. 12, 2023

(30) Foreign Application Priority Data
Feb. 3, 2023 (IN) 202321006920

(51) Int. Cl. G01N 22/00; G01N 33/00; H04Q 9/00

(52) U.S. Cl. CPC G01N 22/00; G01N 33/0075; H04Q 9/00; H04Q 2209/40

(57) ABSTRACT

Provided is a method of monitoring air pollution, the method including: receiving, by a receiver, a wireless communication signal; measuring, by the receiver, attenuation of a millimeter Wave (mm Wave) or terahertz (THz) signal in the received wireless communication signal to obtain a sensing measurement data of the received wireless communication signal; processing the sensing measurement data to obtain air pollution information; and outputting a result of the obtained air pollution information.

### CROSS-REFERENCE TO RELATED APPLICATION(S)

This application is based on and claims priority from Indian Provisional Patent Application No. 202321006920, filed at the Indian Patent Office on Feb. 3, 2023, the disclosure of which is incorporated by reference herein in its entirety.

### FIELD

System and methods consistent with example embodiments of the present disclosure relate to monitoring air pollution, and more specifically, relate to monitoring air pollution in an efficient and accurate manner using integrated sensing and communication.

### BACKGROUND

In general, mobile operators continue to experience an enormous demand for various functionality (e.g., high-speed communication) from a user of an electronic device (e.g., smartphone), which is being driven by multimedia applications and an ever-increasing number of electronic devices connected to a core network (e.g., fifth generation new radio (5G NR) mobile network).

Integrated sensing and communication in 5G systems specified by 3rd Generation Partnership Project (3GPP) is a 5G NR wireless communication system and infrastructure for communication to provide sensing capabilities, and sensing information that may come from radio frequency and/or non-radio frequency based sensors. In a 5G network, base stations are deployed by operators with radio cell planning that allows them to cover a wide area.

Air pollution monitoring is critical for protecting public health and the environment. The air has a direct impact on health, and exposure to certain pollutants can cause a range of health problems, including respiratory and cardiovascular disease, cancer, and other illnesses. Therefore, an accurate and efficient air pollution monitoring is the need of the hour.

Traditionally, the air pollution monitoring equipment mainly includes fixed monitoring stations and mobile monitoring equipment. The current fixed monitoring stations are mainly divided into large fixed monitoring stations (large stations) and small monitoring stations (small stations), whereas mobile monitoring equipment mainly includes special atmospheric environmental monitoring vehicles, drones and handheld devices.

Alternatively, the current air quality monitoring can also be characterized as either professional monitoring systems that are using large number of low-cost air quality monitoring sensors into public transportation or other parts of urban infrastructure. The former provides highly accurate air quality information, but suffers from low spatial resolution, high deployment and maintenance costs. While, the latter suffers from poor accuracy unless sensors are periodically re-calibrated against professional-grade equipment. Currently, carrying out the calibration of the sensors is time-consuming and laborious, limiting the scale at which these types of deployments can operate. These professional monitoring systems are highly expensive and suffer from low spatial resolution while using millions of low-cost sensors results in poor accuracy. Therefore, these specific problems have been experienced with the development of monitoring and tracking of air pollution.

Accordingly, related systems have failed to adequately provide air quality monitoring systems that are inexpensive and address the shortcomings of using millions of low cost sensors throughout a city or an area. Thus, it is desired to address the above-mentioned disadvantages or other shortcomings and provide a useful alternative for air pollution monitoring by using base station and user equipment to monitor air pollution in an efficient and accurate manner.

### SUMMARY

Example embodiments of the present disclosure relate to monitoring air pollution using attenuation of wireless communication signal. Ultimately, example embodiments of the present disclosure eliminate the burden of installing large number of low-cost air quality monitoring sensors into public transportation and carrying out the calibration of the sensors, which are an expensive, time-consuming and laborious.

According to an embodiment, a method is provided. The method of monitoring air pollution includes: receiving, by a receiver, a wireless communication signal; measuring, by the receiver, attenuation of a millimeter Wave (mmWave) or terahertz (THz) signal in the received wireless communication signal to obtain a sensing measurement data of the received wireless communication signal; processing the sensing measurement data to obtain air pollution information; and outputting a result of the obtained air pollution information.

According to an embodiment, a system is provided. The system may be performed by at least one memory storing instructions; and at least one processor configured to execute the instructions to: receive a wireless communication signal; measure attenuation of a millimeter Wave (mmWave) or terahertz (THz) signal in the received wireless communication signal to obtain a sensing measurement data of the received wireless communication signal; process the sensing measurement data to obtain air pollution information; and output a result of the obtained air pollution information.

According to an embodiment, a non-transitory computer-readable recording medium has recorded thereon instructions executable by at least one processor configured to perform a method for monitoring air pollution, the method including: receiving, by a receiver, a wireless communication signal; measuring, by the receiver, attenuation of a millimeter Wave (mmWave) or terahertz (THz) signal in the received wireless communication signal to obtain a sensing measurement data of the received wireless communication signal; processing the sensing measurement data to obtain air pollution information; and outputting a result of the obtained air pollution information.

Additional aspects will be set forth in part in the description that follows and, in part, will be apparent from the description, or may be realized by practice of the presented embodiments of the disclosure.

### BRIEF DESCRIPTION OF THE DRAWINGS

Features, aspects and advantages of certain exemplary embodiments of the disclosure will be described below with reference to the accompanying drawings, in which like reference numerals denote like elements, and wherein:

FIG. 1 illustrates a 5G new radio architecture according to the related art;

FIG. 2 illustrates a system for monitoring air pollution level using integrated sensors and communication;

FIG. 3 illustrates a flowchart of method steps according to an embodiment;

FIG. 4 illustrates a table of example key performance indicators (KPIs) to calculate air pollution information, according to an embodiment;

FIG. 5 illustrates various components according to example embodiment; and

FIG. 6 illustrates a diagram of example components of a device according to an embodiment.

### DETAILED DESCRIPTION

The following detailed description of example embodiments refers to the accompanying drawings.

The foregoing disclosure provides illustration and description, but is not intended to be exhaustive or to limit the implementations to the precise form disclosed. Modifications and variations are possible in light of the above disclosure or may be acquired from practice of the implementations. Further, one or more features or components of one embodiment may be incorporated into or combined with another embodiment (or one or more features of another embodiment). Additionally, in the flowcharts and descriptions of operations provided below, it is understood that one or more operations may be omitted, one or more operations may be added, one or more operations may be performed simultaneously (at least in part), and the order of one or more operations may be switched.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code. It is understood that software and hardware may be designed to implement the systems and/or methods based on the description herein.

Even though particular combinations of features are recited in the claims and/or disclosed in the specification, these combinations are not intended to limit the disclosure of possible implementations. In fact, many of these features may be combined in ways not specifically recited in the claims and/or disclosed in the specification. Although each dependent claim listed below may directly depend on only one claim, the disclosure of possible implementations includes each dependent claim in combination with every other claim in the claim set.

No element, act, or instruction used herein should be construed as critical or essential unless explicitly described as such. Also, as used herein, the articles “a” and “an” are intended to include one or more items, and may be used interchangeably with “one or more.” Where only one item is intended, the term “one” or similar language is used. Also, as used herein, the terms “has,” “have,” “having,” “include,” “including,” or the like are intended to be open-ended terms. Further, the phrase “based on” is intended to mean “based, at least in part, on” unless explicitly stated otherwise. Furthermore, expressions such as “at least one of [A] and [B]” or “at least one of [A] or [B]” are to be understood as including only A, only B, or both A and B.

Example embodiments of the present disclosure provide a method and system for air quality monitoring using integrated sensing and communication. Particularly, it relates to tracking and monitoring air quality based on the attenuation of a wireless communication signal.

Furthermore, the described features, advantages, and characteristics of the present disclosure may be combined in any suitable manner in one or more embodiments. One skilled in the relevant art will recognize, in light of the description herein, that the present disclosure can be practiced without one or more of the specific features or advantages of a particular embodiment. In other instances, additional features and advantages may be recognized in certain embodiments that may not be present in all embodiments of the present disclosure.

Embodiments of the present disclosure are directed to a method and system for monitoring air pollution in an efficient and accurate manner using integrated sensing and communication based on attenuation of wireless communication signals (e.g., Millimeter Wave (mmWave) and terahertz (THz) frequencies used in 5G New Radio (5GNR)).

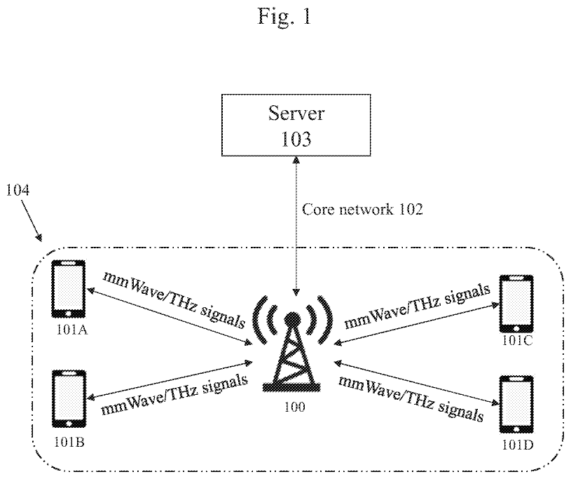

FIG. 1 illustrates a 5G new radio architecture according to the related art. The related system may include user equipment 101A, 101B, 101C and 101D; a base station 100; core network 102; and a server 103.

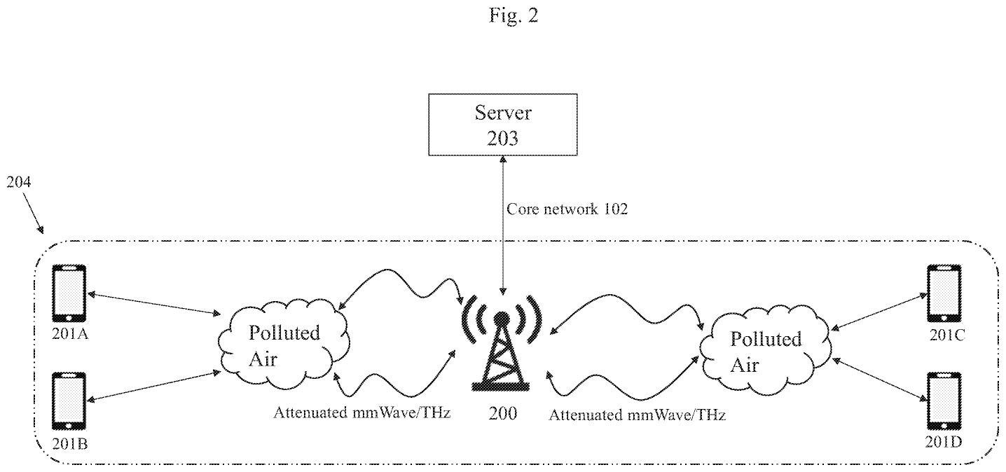

FIG. 2 illustrates a system for monitoring air pollution information using integrated sensors and communication according to an embodiment. The system may include: user equipments 201A, 201B, 201C and 201D; a base station 200; core network 102; and a server 203.

Referring to FIG. 2, wireless communication signal may be received by a receiver that are affected by the absorption wireless communication signals due to air pollutants (e.g., Ozone (O₃), Sulfur dioxide (SO₂), Carbon monoxide (CO), particulate matter (PM), etc.). Further, when the receiver, that may be a transmitter (e.g., antenna, base station 200) or a user equipment 201A, 201B, 201C and 201D (e.g., smart phone, tablet, PDA, etc.), receives attenuated wireless communication signal due to the air pollutants, the receiver may be able to measure air pollution level as they travel from transmitter to the receiver. Particularly, it is already known that different air pollutants absorb wireless communication signals differently due to their respective characteristic properties. Subsequently, due to the air pollutant absorption of the wireless communication signals, the wireless signals are attenuated differently. Consequently, the air pollutants reduce strength of the wireless communication signal that is received by the receiver. Subsequently, the receiver may store the attenuation of wireless communication signal as sensing measurement data either in a storage memory or a cloud server. The sensing measurement data may be processed by the receiver and transmitted to a server 203 or may be processed by the server to obtain air pollution information. The receiver may report the air pollution information to the server 203 via the core network 102. According to an embodiment, the server 203 may be a data center or an application server to output a result of the obtained air pollution information. The application server may be a within a network node or an authorized 3rd party server that may have access to the result of the obtained air pollution information (e.g., at least one of an air quality index (AQI), information or concentration of particulate matter or pollutants in the air, etc.).

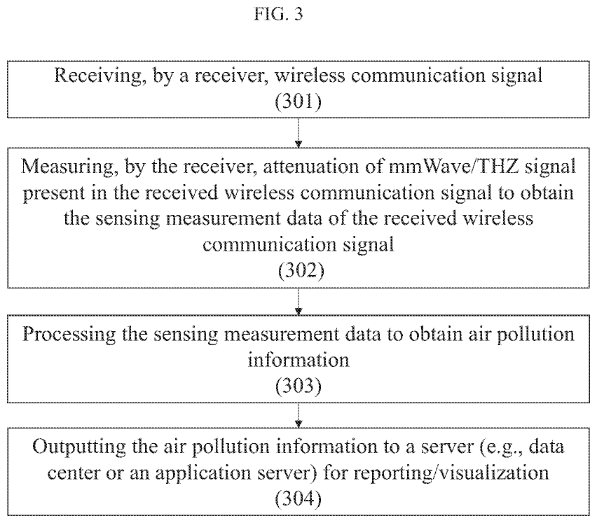

FIG. 3 is a flowchart of a method for monitoring air pollution in an efficient and accurate manner using integrated sensing and communication and attenuation of wireless communication signals. In FIG. 3, operations 301-302 are performed by the receiver and operation 303-304 may be performed either by a server, base station, or a user equipment.

In operation 301, the receiver receives a wireless communication signal. According to an embodiment, the wireless communication signal is 5G communication signal, though it is understood that one or more other embodiments are not limited thereto, and may be applicable to other wireless signals, e.g., mmWave/THz frequency band signal. According to an embodiment, the receiver may be a user equipment or another equipment (e.g., dedicated attenuation measurement device) that receives the wireless communication signal from a base station, or may be a base station that receives the wireless communication signal from user equipment or another device. The receiver may be any device (e.g., PDA, computer, tablet) that is connected to the 5GNR core network and either receives or transmits wireless communication signal to and from a base station.

In operation 302, the receiver measures attenuation of mm Wave/THz signal present in the received wireless communication signal to obtain sensing measurement data. According to an embodiment, the receiver may measure attenuation of mmWave/THz signals present in the wireless communication signal by measuring an absorption coefficient caused by air pollutant and subsequently, measuring the attenuation of received mmWave/THz signals. Further, the receiver may store the attenuation as sensing measurement data either in a memory or a cloud server. Data on absorption of mmWave/THz signals due to various air pollutants is provided by public databases such as Spectraplot, National Institute of Standards and Technology (NIST) and High-Resolution Transmission Molecular Absorption (HITRAN). A process of measuring the attenuation may be as described in “Effects of Major Air Pollutants on Millimeter Wave Spectrum” (Durjan et al., IEEE), incorporated by reference herein in its entirety.

In operation 303, the sensing measurement data is processed by a server, base station or a user equipment or any other equipment (e.g., dedicated attenuation measurement device) to obtain air pollution information (e.g., at least one of an air quality index (AQI), information or concentration of particulate matter or pollutants in the air, etc.). To this end, the receiver may output the sensing measurement data every predetermined time interval (e.g., every second or 60 seconds) to the server to process the sensing measurement data. The air pollution information may be derived or determined from the measured attenuation (e.g., a lookup table or algorithm may be used to correlate the measured attenuation to one or more pollutants or particulate matter). According to an embodiment, the sensing measurement data may be processed by a server. Alternatively, the processing may be carried out within a network node (e.g., within a base station or the receiver). Further, according to an embodiment, the sensing measurement data may include location information corresponding to the sensing measurement data (e.g., at least one of a location of the receiver, a location of a transmitter that transmits the wireless communication signal, a path of the wireless communication signal, etc.). According to an embodiment, the receiver may process the sensing measurement data collected from a plurality of different receivers or transmitted from a plurality of different transmitters (e.g., user equipment). Further, according to an embodiment, the processing may be carried out by an application server that may have access to the sensing measurement data that is measure by the receiver. For example, the application server may be an authorized 3rd party operator or an organization that may utilize the sensing measurement data to generate a result, that is air pollution information based on the sensing measure measurement data.

In operation 304, the server obtains the air pollution information, via the core network (e.g., fifth generation new radio (5GNR) mobile network), associated with the location information and generates a result the outputting of the air pollution information to the server Alternatively, the server may be located within a network node (e.g., within the base station) of a default mobile service provider or network operator. Further, the server may be an application server of an authorized 3rd party. According to an embodiment, the receiver may output the air pollution information to mobile service providers or network operators directly. According to an embodiment, the mobile service provider or network operator may be a default service provider of the receiver or an authorized 3rd party mobile service provider or network operators. When the air pollution is outputted to the server, a result is generated. The result may include, but not limited thereto, air pollution maps, levels of pollution in a certain location, track progress of air pollution, etc. For example, based on the air pollution information including location information for the sensed measurement of attenuation or for the corresponding particulate information, the server may output a visualization of location-based air pollution information. The server may collect the air pollution information from a plurality of different mobile network operators, base stations, etc., and provide the visualization based on this collected information from multiple sources.

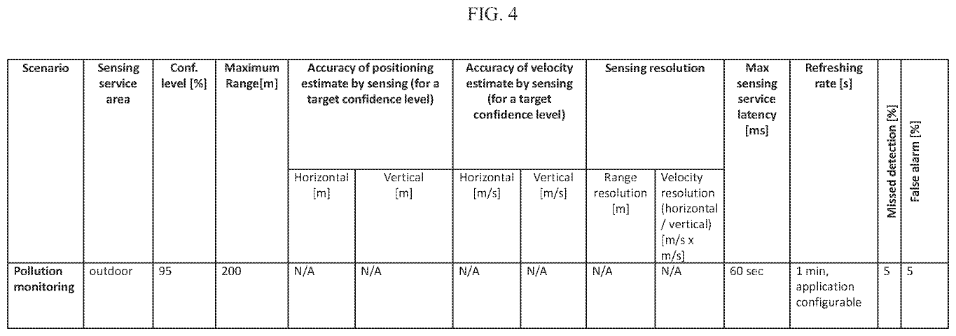

FIG. 4 is a table of example key performance indicators (KPIs) to calculate air pollution information. Particularly, the server may be configured to support sensing services based on KPIs, e.g., it may be configured to define the type of sensing service areas (e.g., outdoor, indoor, etc.), confidence level (e.g., 95%), a maximum range of the sensing measurement data (e.g., 200 meters), a maximum sensing service latency (e.g., 60 seconds), refreshing rate (e.g., 1 minute), missed detection (e.g., 5%), and false alarms (e.g., 5%). Alternatively, the server may be configured to calculate other KPIs and change the values of aforementioned KPIs to provide an accurate and efficient air pollution monitoring system based on the one or more embodiments of the present disclosure. The server may provide the configurations to the air pollution monitoring system (e.g., receiver), which may thereafter perform the above-described operations according to the configurations.

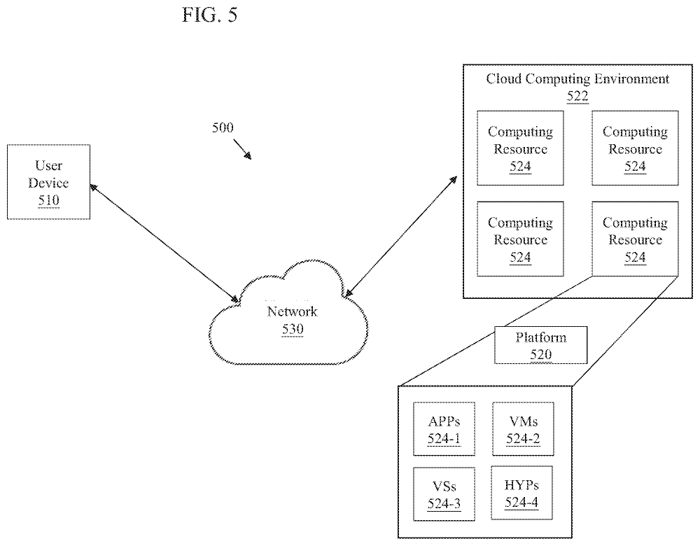

FIG. 5 is a diagram of an example environment 500 in which systems and/or methods, described herein, may be implemented. As shown in FIG. 5, environment 500 may include a user device 510, a platform 520, and a network 530. Devices of environment 500 may interconnect via wired connections, wireless connections, or a combination of wired and wireless connections. In embodiments, any of the functions and operations described with reference to FIG. 2 above may be performed by any combination of elements illustrated in FIG. 5.

User device 510 includes one or more devices capable of receiving, generating, storing, processing, and/or providing information associated with platform 520. For example, user device 710 may include a computing device (e.g., a desktop computer, a laptop computer, a tablet computer, a handheld computer, a smart speaker, a server, etc.), a mobile phone (e.g., a smart phone, a radiotelephone, etc.), a wearable device (e.g., a pair of smart glasses or a smart watch), or a similar device. In some implementations, user device 510 may receive information from and/or transmit information to platform 520.

Platform 520 includes one or more devices capable of receiving, generating, storing, processing, and/or providing information. In some implementations, platform 520 may include a cloud server or a group of cloud servers. In some implementations, platform 520 may be designed to be modular such that certain software components may be swapped in or out depending on a particular need. As such, platform 520 may be easily and/or quickly reconfigured for different uses.

In some implementations, as shown, platform 520 may be hosted in cloud computing environment 522. Notably, while implementations described herein describe platform 520 as being hosted in cloud computing environment 522, in some implementations, platform 520 may not be cloud-based (i.e., may be implemented outside of a cloud computing environment) or may be partially cloud-based.

Cloud computing environment 522 includes an environment that hosts platform 520. Cloud computing environment 522 may provide computation, software, data access, storage, etc., services that do not require end-user (e.g., user device 510) knowledge of a physical location and configuration of system(s) and/or device(s) that hosts platform 520. As shown, cloud computing environment 522 may include a group of computing resources 524 (referred to collectively as “computing resources 524” and individually as “computing resource 524”).

Computing resource 524 includes one or more personal computers, a cluster of computing devices, workstation computers, server devices, or other types of computation and/or communication devices. In some implementations, computing resource 524 may host platform 520. The cloud resources may include compute instances executing in computing resource 524, storage devices provided in computing resource 524, data transfer devices provided by computing resource 524, etc. In some implementations, computing resource 724 may communicate with other computing resources 524 via wired connections, wireless connections, or a combination of wired and wireless connections.

As further shown in FIG. 5, computing resource 524 includes a group of cloud resources, such as one or more applications (“APPs”) 524-1, one or more virtual machines (“VMs”) 524-2, virtualized storage (“VSs”) 524-3, one or more hypervisors (“HYPs”) 524-4, or the like.

Application 524-1 includes one or more software applications that may be provided to or accessed by user device 510. Application 524-1 may eliminate a need to install and execute the software applications on user device 510. For example, application 524-1 may include software associated with platform 520 and/or any other software capable of being provided via cloud computing environment 522. In some implementations, one application 524-1 may send/receive information to/from one or more other applications 524-1, via virtual machine 524-2.

Virtual machine 524-2 includes a software implementation of a machine (e.g., a computer) that executes programs like a physical machine. Virtual machine 524-2 may be either a system virtual machine or a process virtual machine, depending upon use and degree of correspondence to any real machine by virtual machine 524-2. A system virtual machine may provide a complete system platform that supports execution of a complete operating system (“OS”). A process virtual machine may execute a single program, and may support a single process. In some implementations, virtual machine 524-2 may execute on behalf of a user (e.g., user device 510), and may manage infrastructure of cloud computing environment 522, such as data management, synchronization, or long-duration data transfers.

Virtualized storage 524-3 includes one or more storage systems and/or one or more devices that use virtualization techniques within the storage systems or devices of computing resource 524. In some implementations, within the context of a storage system, types of virtualizations may include block virtualization and file virtualization. Block virtualization may refer to abstraction (or separation) of logical storage from physical storage so that the storage system may be accessed without regard to physical storage or heterogeneous structure. The separation may permit administrators of the storage system flexibility in how the administrators manage storage for end users. File virtualization may eliminate dependencies between data accessed at a file level and a location where files are physically stored. This may enable optimization of storage use, server consolidation, and/or performance of non-disruptive file migrations.

Hypervisor 524-4 may provide hardware virtualization techniques that allow multiple operating systems (e.g., “guest operating systems”) to execute concurrently on a host computer, such as computing resource 524. Hypervisor 524-4 may present a virtual operating platform to the guest operating systems, and may manage the execution of the guest operating systems. Multiple instances of a variety of operating systems may share virtualized hardware resources.

Network 530 includes one or more wired and/or wireless networks. For example, network 530 may include a cellular network (e.g., a fifth generation (5G) network, a long-term evolution (LTE) network, a third generation (3G) network, a code division multiple access (CDMA) network, etc.), a public land mobile network (PLMN), a local area network (LAN), a wide area network (WAN), a metropolitan area network (MAN), a telephone network (e.g., the Public Switched Telephone Network (PSTN)), a private network, an ad hoc network, an intranet, the Internet, a fiber optic-based network, or the like, and/or a combination of these or other types of networks.

The number and arrangement of devices and networks shown in FIG. 5 are provided as an example. In practice, there may be additional devices and/or networks, fewer devices and/or networks, different devices and/or networks, or differently arranged devices and/or networks than those shown in FIG. 5. Furthermore, two or more devices shown in FIG. 5 may be implemented within a single device, or a single device shown in FIG. 5 may be implemented as multiple, distributed devices. Additionally, or alternatively, a set of devices (e.g., one or more devices) of environment 500 may perform one or more functions described as being performed by another set of devices of environment 500.

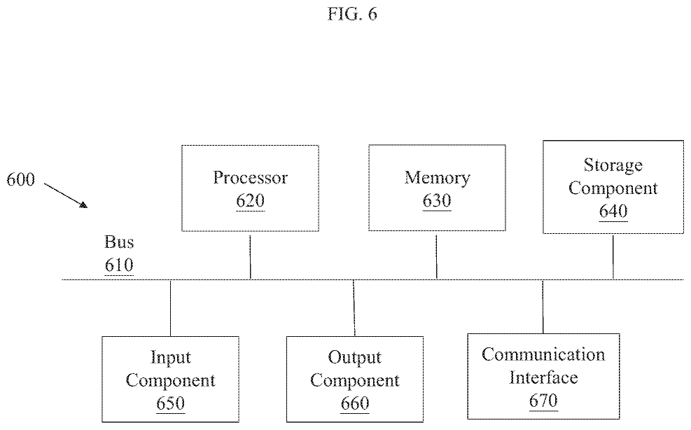

FIG. 6 is a diagram of example components of a device 600. Device 600 may correspond to user device 510 and/or platform 520. As shown in FIG. 6, device 600 may include a bus 610, a processor 620, a memory 630, a storage component 640, an input component 650, an output component 660, and a communication interface 670.

Bus 610 includes a component that permits communication among the components of device 600. Processor 620 may be implemented in hardware, firmware, or a combination of hardware and software. Processor 620 may be a central processing unit (CPU), a graphics processing unit (GPU), an accelerated processing unit (APU), a microprocessor, a microcontroller, a digital signal processor (DSP), a field-programmable gate array (FPGA), an application-specific integrated circuit (ASIC), or another type of processing component. In some implementations, processor 620 includes one or more processors capable of being programmed to perform a function. Memory 630 includes a random access memory (RAM), a read only memory (ROM), and/or another type of dynamic or static storage device (e.g., a flash memory, a magnetic memory, and/or an optical memory) that stores information and/or instructions for use by processor 620.

Storage component 640 stores information and/or software related to the operation and use of device 600. For example, storage component 640 may include a hard disk (e.g., a magnetic disk, an optical disk, a magneto-optic disk, and/or a solid state disk), a compact disc (CD), a digital versatile disc (DVD), a floppy disk, a cartridge, a magnetic tape, and/or another type of non-transitory computer-readable medium, along with a corresponding drive. Input component 850 includes a component that permits device 600 to receive information, such as via user input (e.g., a touch screen display, a keyboard, a keypad, a mouse, a button, a switch, and/or a microphone). Additionally, or alternatively, input component 650 may include a sensor for sensing information (e.g., a global positioning system (GPS) component, an accelerometer, a gyroscope, and/or an actuator). Output component 660 includes a component that provides output information from device 600 (e.g., a display, a speaker, and/or one or more light-emitting diodes (LEDs)).

Communication interface 670 includes a transceiver-like component (e.g., a transceiver and/or a separate receiver and transmitter) that enables device 600 to communicate with other devices, such as via a wired connection, a wireless connection, or a combination of wired and wireless connections. Communication interface 670 may permit device 600 to receive information from another device and/or provide information to another device. For example, communication interface 670 may include an Ethernet interface, an optical interface, a coaxial interface, an infrared interface, a radio frequency (RF) interface, a universal serial bus (USB) interface, a Wi-Fi interface, a cellular network interface, or the like.

Device 600 may perform one or more processes described herein. Device 600 may perform these processes in response to processor 620 executing software instructions stored by a non-transitory computer-readable medium, such as memory 630 and/or storage component 640. A computer-readable medium is defined herein as a non-transitory memory device. A memory device includes memory space within a single physical storage device or memory space spread across multiple physical storage devices.

Software instructions may be read into memory 630 and/or storage component 640 from another computer-readable medium or from another device via communication interface 670. When executed, software instructions stored in memory 630 and/or storage component 640 may cause processor 620 to perform one or more processes described herein.

Additionally, or alternatively, hardwired circuitry may be used in place of or in combination with software instructions to perform one or more processes described herein. Thus, implementations described herein are not limited to any specific combination of hardware circuitry and software.

The number and arrangement of components shown in FIG. 6 are provided as an example. In practice, device 600 may include additional components, fewer components, different components, or differently arranged components than those shown in FIG. 6. Additionally, or alternatively, a set of components (e.g., one or more components) of device 600 may perform one or more functions described as being performed by another set of components of device 600.

In embodiments, any one of the operations or processes of FIGS. 2 and 3 may be implemented by or using any one of the elements illustrated in FIGS. 5 and 6. It is understood that other embodiments are not limited thereto, and may be implemented in a variety of different architectures (e.g., bare metal architecture, any cloud-based architecture or deployment architecture such as Kubernetes, Docker, OpenStack, etc.).

The foregoing disclosure provides illustration and description, but is not intended to be exhaustive or to limit the implementations to the precise form disclosed. Modifications and variations are possible in light of the above disclosure or may be acquired from practice of the implementations.

Some embodiments may relate to a system, a method, and/or a computer readable medium at any possible technical detail level of integration. Further, one or more of the above components described above may be implemented as instructions stored on a computer readable medium (or media) and executable by at least one processor (and/or may include at least one processor). The computer readable medium may include a computer-readable non-transitory storage medium (or media) having computer readable program instructions thereon for causing a processor to carry out operations. The at least one processor may be distributed across a plurality of devices (e.g., a user equipment, a base station, and a server) that respectively execute instructions stored on the media that is similarly distributed across the plurality of devices.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program code/instructions for carrying out operations may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects or operations.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer readable media according to various embodiments. In this regard, each block in the flowchart or block diagrams may represent a microservice(s), module, segment, or portion of instructions, which comprises one or more executable instructions for implementing the specified logical function(s). The method, computer system, and computer readable medium may include additional blocks, fewer blocks, different blocks, or differently arranged blocks than those depicted in the Figures. In some alternative implementations, the functions noted in the blocks may occur out of the order noted in the Figures. For example, two blocks shown in succession may, in fact, be executed concurrently or substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

It will be apparent that systems and/or methods, described herein, may be implemented in different forms of hardware, firmware, or a combination of hardware and software. The actual specialized control hardware or software code used to implement these systems and/or methods is not limiting of the implementations. Thus, the operation and behavior of the systems and/or methods were described herein without reference to specific software code—it being understood that software and hardware may be designed to implement the systems and/or methods based on the description herein.

Various further respective aspects and features of embodiments of the present disclosure may be defined by the following items:

Item [1]: A method of monitoring air pollution, the method including receiving, by a receiver, a wireless communication signal; measuring, by the receiver, attenuation of a millimeter Wave (mmWave) or terahertz (THz) signal in the received wireless communication signal to obtain a sensing measurement data of the received wireless communication signal; processing the sensing measurement data to obtain air pollution information; and outputting a result of the obtained air pollution information.

Item [2]: The method of item [1], wherein the outputting includes: transmitting the air pollution information to a server; analyzing, by the server, a plurality of air pollution information obtained by measuring attenuation of mmWave or THz signals in a plurality of wireless communication signals; and generating a report on air pollution based on the analyzing.

Item [3]: The method of any one of items [1] to [2], wherein: the receiver is a user equipment that receives the wireless communication signal from a base station; or the receiver is the base station that receives the wireless communication signal from the user equipment.

Item [4]: The method of any one of items [1] to [3], wherein the processing includes processing, by the receiver, the sensing measurement data to obtain the air pollution information.

Item [5]: The method of any one of items [1] to [4], wherein the processing includes: transmitting, by the receiver, the sensing measurement data to a server; and processing, by the server, the sensing measurement data to obtain the air pollution information.

Item [6]: The method of item [5], wherein: the transmitting includes transmitting a plurality of sensing measurement data to the server at predetermined time intervals; and the processing by the server includes processing, by the server, the plurality of sensing measurement data to obtain the air pollution information.

Item [7]: The method of any one of items [1] to [6], wherein: the processing includes processing a plurality of sensing measurement data that is obtained by measuring attenuation of mmWave or THz signals in a plurality of wireless communication signals, to obtain the air pollution information; and the plurality of wireless communication signals are transmitted by a plurality of user equipment to a base station that measures the attenuation, or are transmitted by the base station to the plurality of user equipment that measure the attenuation.

Item [8]: The method of any one of items [1] to [7], wherein the sensing measurement data includes a measurement of the attenuation, and at least one of location information corresponding to the sensing measurement data and a path of the wireless communication signal.

Item [9]: A system for monitoring air pollution, the system including: at least one memory storing instructions; and at least one processor configured to execute the instructions to: receive a wireless communication signal; measure attenuation of a millimeter Wave (mmWave) or terahertz (THz) signal in the received wireless communication signal to obtain a sensing measurement data of the received wireless communication signal; process the sensing measurement data to obtain air pollution information; and output a result of the obtained air pollution information.

Item [10]: The system of item [9], wherein the at least one processor is further configured to execute the instructions to output the result by: analyzing a plurality of air pollution information obtained by measuring attenuation of mmWave or THz signals in a plurality of wireless communication signals; and generating a report on air pollution based on the analyzing.

Item [11]: The system of any one of items [9] to [10], wherein: the wireless communication signal is received, from a base station, by a user equipment that performs the measuring; or the wireless communication signal is received, from the user equipment, by the base station that performs the measuring.

Item [12]: The system of any one of items [9] to [11], wherein the at least one processor is further configured to execute the instructions to: process a plurality of sensing measurement data obtained by measuring attenuation of mmWave or THz signals in a plurality of wireless communication signals, to obtain the air pollution information.

Item [13]: The system of item [12], wherein: the plurality of wireless communication signals are transmitted by a plurality of user equipment to a base station that measures the attenuation, or are transmitted by the base station to the plurality of user equipment that measure the attenuation.

Item [14]: The system of any one of items [9] to [13], wherein the sensing measurement data includes a measurement of the attenuation, and at least one of location information corresponding to the sensing measurement data and a path of the wireless communication signal.

Item [15]: At least one non-transitory computer-readable recording medium having recorded thereon instructions executable by at least one processor to perform a method of monitoring air pollution, the method including: receiving, by a receiver, a wireless communication signal; measuring, by the receiver, attenuation of a millimeter Wave (mmWave) or terahertz (THz) signal in the received wireless communication signal to obtain a sensing measurement data of the received wireless communication signal; processing the sensing measurement data to obtain air pollution information; and outputting a result of the obtained air pollution information.

Item [16]: The at least one non-transitory computer-readable recording medium of item [15], wherein the outputting includes: analyzing a plurality of air pollution information obtained by measuring attenuation of mmWave or THz signals in a plurality of wireless communication signals; and generating a report on air pollution based on the analyzing.

Item [17]: The at least one non-transitory computer-readable recording medium of any one of items to [16], wherein: the receiver is a user equipment that receives the wireless communication signal from a base station; or the receiver is the base station that receives the wireless communication signal from the user equipment.

Item [18]: The at least one non-transitory computer-readable recording medium of any one of items to [17], wherein the processing includes: processing a plurality of sensing measurement data obtained by measuring attenuation of mm Wave or THz signals in a plurality of wireless communication signals, to obtain the air pollution information.

Item [19]: The at least one non-transitory computer-readable recording medium of any one of items to [18], wherein the plurality of wireless communication signals are transmitted by a plurality of user equipment to a base station that measures the attenuation, or are transmitted by the base station to the plurality of user equipment that measure the attenuation.

Item [20]: The at least one non-transitory computer-readable recording medium of any one of items to [19], wherein the sensing measurement data includes a measurement of the attenuation, and at least one of location information corresponding to the sensing measurement data and a path of the wireless communication signal.

It can be understood that numerous modifications and variations of the present disclosure are possible in light of the above teachings. It will be apparent that within the scope of the appended clauses, the present disclosures may be practiced otherwise than as specifically described herein.

What is claimed is:

1. A method of monitoring air pollution, the method comprising:
receiving, by a receiver, a wireless communication signal;
measuring, by the receiver, attenuation of a millimeter Wave (mmWave) or terahertz (THz) signal in the received wireless communication signal to obtain a sensing measurement data of the received wireless communication signal;
processing the sensing measurement data to obtain air pollution information; and
outputting a result of the obtained air pollution information.

2. The method of claim 1, wherein the outputting comprises:
transmitting the air pollution information to a server;
analyzing, by the server, a plurality of air pollution information obtained by measuring attenuation of mmWave or THz signals in a plurality of wireless communication signals; and
generating a report on air pollution based on the analyzing.

3. The method of claim 1, wherein:
the receiver is a user equipment that receives the wireless communication signal from a base station; or
the receiver is the base station that receives the wireless communication signal from the user equipment.

4. The method of claim 1, wherein the processing comprises processing, by the receiver, the sensing measurement data to obtain the air pollution information.

5. The method of claim 1, wherein the processing comprises:
transmitting, by the receiver, the sensing measurement data to a server; and
processing, by the server, the sensing measurement data to obtain the air pollution information.

6. The method of claim 5, wherein:
the transmitting comprises transmitting a plurality of sensing measurement data to the server at predetermined time intervals; and
the processing by the server comprises processing, by the server, the plurality of sensing measurement data to obtain the air pollution information.

7. The method of claim 1, wherein:
the processing comprises processing a plurality of sensing measurement data that is obtained by measuring attenuation of mm Wave or THz signals in a plurality of wireless communication signals, to obtain the air pollution information; and
the plurality of wireless communication signals are transmitted by a plurality of user equipment to a base station that measures the attenuation, or are transmitted by the base station to the plurality of user equipment that measure the attenuation.

8. The method of claim 1, wherein the sensing measurement data comprises a measurement of the attenuation, and at least one of location information corresponding to the sensing measurement data and a path of the wireless communication signal.

9. A system for monitoring air pollution, the system comprising:
at least one memory storing instructions; and
at least one processor configured to execute the instructions to:
receive a wireless communication signal;
measure attenuation of a millimeter Wave (mmWave) or terahertz (THz) signal in the received wireless communication signal to obtain a sensing measurement data of the received wireless communication signal;
process the sensing measurement data to obtain air pollution information; and
output a result of the obtained air pollution information.

10. The system of claim 9, wherein the at least one processor is further configured to execute the instructions to output the result by:
analyzing a plurality of air pollution information obtained by measuring attenuation of mm Wave or THz signals in a plurality of wireless communication signals; and
generating a report on air pollution based on the analyzing.

11. The system of claim 9, wherein:
the wireless communication signal is received, from a base station, by a user equipment that performs the measuring; or
the wireless communication signal is received, from the user equipment, by the base station that performs the measuring.

12. The system of claim 9, wherein the at least one processor is further configured to execute the instructions to:
process a plurality of sensing measurement data obtained by measuring attenuation of mm Wave or THz signals in a plurality of wireless communication signals, to obtain the air pollution information.

13. The system of claim 12, wherein:
the plurality of wireless communication signals are transmitted by a plurality of user equipment to a base station that measures the attenuation, or are transmitted by the base station to the plurality of user equipment that measure the attenuation.

14. The system of claim 9, wherein the sensing measurement data comprises a measurement of the attenuation, and at least one of location information corresponding to the sensing measurement data and a path of the wireless communication signal.

15. At least one non-transitory computer-readable recording medium having recorded thereon instructions executable by at least one processor to perform a method of monitoring air pollution, the method comprising:
receiving, by a receiver, a wireless communication signal;
measuring, by the receiver, attenuation of a millimeter Wave (mmWave) or terahertz (THz) signal in the received wireless communication signal to obtain a sensing measurement data of the received wireless communication signal;
processing the sensing measurement data to obtain air pollution information; and
outputting a result of the obtained air pollution information.

16. The at least one non-transitory computer-readable recording medium of claim 15, wherein the outputting comprises:
analyzing a plurality of air pollution information obtained by measuring attenuation of mmWave or THz signals in a plurality of wireless communication signals; and
generating a report on air pollution based on the analyzing.

17. The at least one non-transitory computer-readable recording medium of claim 15, wherein:
the receiver is a user equipment that receives the wireless communication signal from a base station; or
the receiver is the base station that receives the wireless communication signal from the user equipment.

18. The at least one non-transitory computer-readable recording medium of claim 15, wherein the processing comprises:
processing a plurality of sensing measurement data obtained by measuring attenuation of mmWave or THz signals in a plurality of wireless communication signals, to obtain the air pollution information.

19. The at least one non-transitory computer-readable recording medium of claim 18, wherein the plurality of wireless communication signals are transmitted by a plurality of user equipment to a base station that measures the attenuation, or are transmitted by the base station to the plurality of user equipment that measure the attenuation.

20. The at least one non-transitory computer-readable recording medium of claim 15, wherein the sensing measurement data comprises a measurement of the attenuation, and at least one of location information corresponding to the sensing measurement data and a path of the wireless communication signal.
